(19) United States
(12) Patent Application Publication
(10) Pub. No.: US 2024/0367164 A1
(43) Pub. Date: Nov. 7, 2024

(54) SYSTEMS AND METHODS FOR CONTROLLING FLUID FLOW BETWEEN MULTIPLE CHAMBERS OF A TESTING DEVICE

(71) Applicant: President and Fellows of Harvard College, Cambridge, MA (US)

(72) Inventors: Thomas E. Schaus, Cambridge, MA (US); Peng Yin, Cambridge, MA (US)

(21) Appl. No.: 18/690,217

(22) PCT Filed: Sep. 7, 2022
(86) PCT No.: PCT/US22/42751

§ 371 Date: Mar. 7, 2024

Related U.S. Application Data
(60) Provisional application No. 63/241,868, filed on Sep. 8, 2021.

(51) Int. Cl. B01L 3/00

(52) U.S. Cl. CPC B01L 3/502; B01L 2200/0684; B01L 2200/0689; B01L 2200/16; B01L 2300/042; B01L 2300/161

(57) ABSTRACT

A device for performing an assay includes a housing, an elongated member, and a vent. The housing has a first end and a second end. The housing defines a first opening at the first end, a first chamber, and a second chamber. The first chamber is fluidly connected to (i) the second chamber, and (ii) an exterior of the housing via the first opening. The elongated member is configured to be received through the first opening such that the elongated member is least partially disposed within the first chamber. The vent is configured to aid in controlling flow of a fluid from the first chamber of the housing to the second chamber of the housing.

### CROSS-REFERENCE TO RELATED APPLICATIONS

This application claims the benefit of and priority to U.S. Provisional Patent Application No. 63/241,868 filed Sep. 8, 2021, which is hereby incorporated by reference herein in its entirety.

### STATEMENT REGARDING FEDERALLY SPONSORED RESEARCH

This invention was made with government support under GM133052 awarded by the National Institutes of Health. The government has certain rights in the invention.

### TECHNICAL FIELD

The present disclosure relates generally to devices and methods for performing tests on samples. Specifically, the present disclosure is directed to a testing device with multiple chambers that advances fluids between a first chamber and a second chamber of the testing device.

### BACKGROUND

Laboratory process in biotechnology are typically complex and require high levels of training and environmental control. For example, reactions where DNA is amplified (e.g., copied exponentially) often involve complicated mixing steps, heating steps, liquid transfer steps, etc. It can be difficult to precisely control reactions such as these to ensure accurate measurements. The present disclosure is related to testing devices that allow for the precise control of the flow of fluid between various chambers of a testing device.

### SUMMARY

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to some implementations of the present disclosure, a device for performing an assay includes a housing, an elongated member, and a vent. The housing has a first end and a second end. The housing defines a first opening at the first end, a first chamber, and a second chamber. The first chamber is fluidly connected to (i) the second chamber and (ii) an exterior of the housing via the first opening. The elongated member is configured to be received through the first opening such that the elongated member is least partially disposed within the first chamber. The vent is configured to aid in controlling flow of a fluid from the first chamber of the housing to the second chamber of the housing.

According to some implementations of the present disclosure, a device for performing an assay includes a housing, an elongated member, and a vent. The housing has a first end and a second end. The housing defines a first opening at the first end, a first chamber, and a second chamber. The first chamber is fluidly connected to (i) the second chamber and (ii) an exterior of the housing via the first opening. The elongated member is configured to be received through the first opening such that the elongated member is least partially disposed within the first chamber. The elongated member aids in generating an air pressure in the first chamber and the second chamber. The vent is configured to aid in controlling flow of a fluid from the first chamber of the housing to the second chamber of the housing. The vent can be activated to release the air pressure generated in the first chamber and the second chamber, to thereby cause the fluid to flow from the first chamber to the second chamber.

According to some implementations of the present disclosure, a system for performing an assay includes a device and a base station. The device includes a housing, an elongated member, and a vent. The housing has a first end and a second end. The housing defines a first opening at the first end, a first chamber, and a second chamber. The first chamber is fluidly connected to (i) the second chamber and (ii) an exterior of the housing via the first opening. The elongated member is configured to be received through the first opening such that the elongated member is least partially disposed within the first chamber. The vent is configured to aid in controlling flow of a fluid from the first chamber of the housing to the second chamber of the housing.

According to some implementations of the present disclosure, a system for performing an assay includes a device and a base station. The device includes a housing, an elongated member, and a vent. The housing has a first end and a second end. The housing defines a first opening at the first end, a first chamber, and a second chamber. The first chamber is fluidly connected to (i) the second chamber and (ii) an exterior of the housing via the first opening. The elongated member is configured to be received through the first opening such that the elongated member is least partially disposed within the first chamber. The elongated member aids in generating an air pressure in the first chamber and the second chamber. The vent is configured to aid in controlling flow of a fluid from the first chamber of the housing to the second chamber of the housing. The vent can be activated to release the air pressure generated in the first chamber and the second chamber, to thereby cause the fluid to flow from the first chamber to the second chamber.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments, and are therefore not to be considered as limitations on the scope of the various embodiments or claims.

FIG. 1 illustrates a first testing device for performing one or more assays, according to some implementations of the present disclosure;

FIG. 2 illustrates a second testing device for performing one or more assays, according to some implementations of the present disclosure;

FIG. 3A illustrates a first step in a sequence of using the testing device of FIG. 1, according to some implementations of the present disclosure;

FIG. 3B illustrates a second step in the sequence of using the testing device of FIG. 1, according to some implementations of the present disclosure;

FIG. 3C illustrates a third step in the sequence of using the testing device of FIG. 1, according to some implementations of the present disclosure;

FIG. 3D illustrates a fourth step in the sequence of using the testing device of FIG. 1, according to some implementations of the present disclosure;

FIG. 3E illustrates a fifth step in the sequence of using the testing device of FIG. 1, according to some implementations of the present disclosure;

FIG. 3F illustrates a sixth step in the sequence of using the testing device of FIG. 1, according to some implementations of the present disclosure;

FIG. 4A illustrates a first technique for adding a liquid reagent to a testing device, according to some implementations of the present disclosure;

FIG. 4B illustrates a first technique for adding a liquid reagent to a testing device, according to some implementations of the present disclosure;

FIG. 5A illustrates a first arrangement of chambers within a testing device, according to some implementations of the present disclosure;

FIG. 5B illustrates a second arrangement of chambers within a testing device, according to some implementations of the present disclosure; and

FIG. 6 illustrates the use of different types of lypholization beads within a testing device, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure provide a simple, inexpensive system and method of advancing biochemical reactions that may require multiple sequential compartments or chambers, whether due to different reagents, temperatures, or other requirements. In some implementations, air pressure can be generated within the chambers. The air pressure can be released to cause fluid to flow between chambers.

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

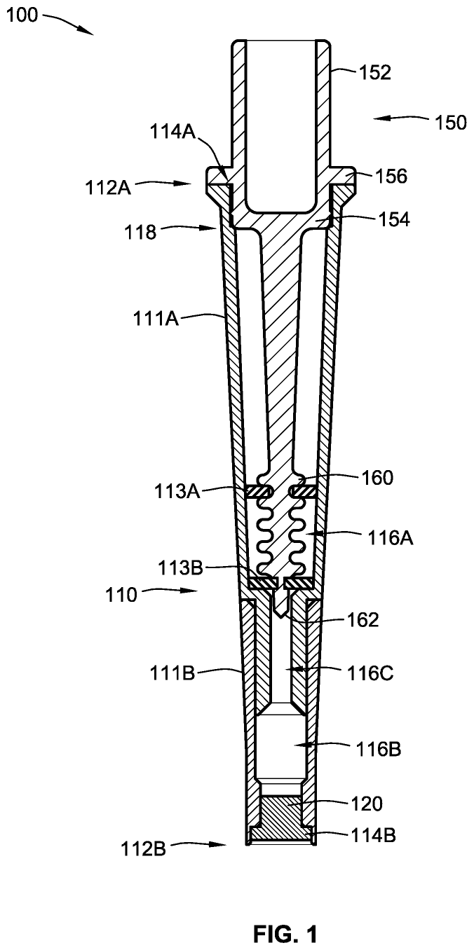

FIG. 1 is a cross-sectional view of a testing device 100, according to some implementations of the present disclosure. The testing device 100 can allow advancing reactions from one chamber of the testing device 100 to another chamber of the testing device 100. The testing device 100 can be an inexpensive, disposable device. The testing device 100 includes a housing 110 that defines a number of chambers, passageways, openings, etc. In the illustrated implementation, the housing 110 has a first end 112A and a second end 112B. The housing 110 includes a first opening 114A adjacent to the first end 112A, and a second opening 114B adjacent to the second end 112B. The housing further defines a first chamber 116A and a second chamber 116B. The first chamber 116A is defined between the first opening 114A and the second chamber 116B. The second chamber 116B is defined between the first chamber 116A and the second opening 114B. In the illustrated implementation, the first chamber 116A and the second chamber 116B are connected to each other, such that there is no physical structure of the housing 110 prevents fluid (such as a liquid, a liquid-based mixture, a gas, etc.)

from flowing between the first chamber 116A and the second chamber 116B.

In the illustrated implementation, the housing 110 is formed from two separate housing portions 111A and 111B that can be coupled together, for example via a friction fit, or other coupling mechanisms or techniques. The first opening 114A and the first chamber 116A are defined by the housing portion 111A. The second opening 114B and the second chamber 116B are defined by the housing portion 111B. The open end of the housing portion 111A that is opposite from the first opening 114A is received by an open end of the housing portion 111B that is opposite from the second opening 114B, so that the first chamber 116A and the second chamber 116B are fluidly connected. In other implementations, the housing 110 may be formed from a single unitary piece. As used herein, the term “housing” refers to both implementations where the housing is formed as a single unitary piece, and implementations where the housing is formed from multiple pieces that are coupled together.

The device 100 includes a vent that aids in controlling flow of a fluid from the first chamber 116A to the second chamber 116B. Generally, the vent can be any structure or combination of structures that can be activated to prevent and allow fluid flow from the first chamber 116A to the second chamber 116B. In the illustrated implementation, the vent includes the second opening 114B, and a plug 120 that is disposed within the second opening 114B. The plug 120 can be formed from a porous hydrophobic material, such that air and other gases can pass through the plug 120, but liquids cannot pass through the plug 120.

The device 100 further includes an elongated member 150 that can be inserted into the housing 110 of the device 100. As shown, the elongated member 150 can be received through the first opening 114A of the housing 110, such that at least a portion of the elongated member 150 is disposed in the first chamber 116A. In the illustrated implementation, the elongated member 150 is a sample swab, and has a handle 152 and a sample collection head. The sample collection head is formed from a plurality of radially-extending ribs 160, and an axially-extending tip 162. The diameter of the elongated member 150 decreases abruptly after the point along the elongated member 150 that is disposed within the housing 110, such that a circumferential shoulder 154 is formed near the handle 152. The elongated member 150 also includes a circumferential flange 156 extending from the handle 152.

When the elongated member 150 is inserted into the housing 110, the shoulder 154 and the sample collection head are disposed within the first chamber 116A. The shoulder 154 is positioned near the first end 112A of the housing 110, while the sample collection head is positioned near the intersection between the first chamber 116A and the second chamber 116B (e.g., near the intersection between housing portion 111A and housing portion 111B).

The shoulder 154 and the flange 156 act as sealing members that aid in sealing the first opening 114A when the elongated member 150 is inserted into the housing 110. As shown in FIG. 1, the shoulder 154 extends into a shallow circumferential depression 118 that is defined in the interior of the housing 110, near the first end 112A. In some implementations, the housing 110 and/or the elongated member 150 are formed from resilient materials, such that the shoulder 154 is configured to snap into the depression 118 when the elongated member 150 is inserted. The flange 156 contacts the outside of the housing 110 at the first end 112A. The diameter of the flange 156 is generally larger than the diameter of the first opening 114A, and thus the flange 156 covers the first opening 114A. The contact between the shoulder 154 and the depression 118 on the interior of the housing 110, and the contact between the flange 156 and the outside of the housing 110, form a seal that prevents liquid and air from entering or exiting the housing 110 through the first opening 114A.

During use of the device 100, the elongated member 150 can be used to collect a sample. For example, the elongated member 150 can be used as a nasal swab to collect a sample from a person's nose. The elongated member 150 can then be inserted into the device 100, so that the sample (which is generally collected specifically by the sample collection head), is disposed in the first chamber 116A. The device may include a number of different substances disposed in the first chamber 116A, the second chamber 116B, or both, that are used to perform a desired assay (e.g., a desired test) on the sample. For example, the first chamber 116A and the second chamber 116B could include one or more reagents and/or one or more buffers that cause a reaction to occur when the sample is added. The first chamber 116A and the second chamber 116B could also include substances configured to aid in mixing the sample with another substance.

After a sample has been collected by the elongated member 150, the elongated member 150 is inserted into the housing 110. Generally, the device 100 will be located in some type of base station (or other holding mechanism) that includes a component configured to seal the second opening 114B of the device. In the illustrated implementation, this seal is formed by covering the plug 120, which prevents air from escaping the inside of the housing 110 through the plug 120 (because the plug 120 is porous). The presence of the elongated member 150 within the housing 110 decreases the internal volume of the housing 110. Because the housing 110 is sealed near the first end 112A by the shoulder 154 and the flange 156 of the elongated member 150, and near the second end 112B by the base station, an increased air pressure is generated within the housing 110 in response to the elongated member 150 being inserted into the housing 110. This air pressure is generated between (i) the sealing members of the elongated member 150 (e.g., the shoulder 154 and/or the flange 156), and (ii) the second opening 114B and the plug 120.

The sample (collected by the sample collection head) will generally be positioned in the first chamber 116A, along with any reagents or other substances residing in the first chamber 116A. Generally, a liquid reagent is disposed in the first chamber 116A is a liquid. Because the fluid in the first chamber 116A (formed from the sample and the liquid reagents or other substances) generally exists in very small quantities, gravity does not exert a significant amount of force on the fluid. Thus, even though the first chamber 116A and the second chamber 116B are fluidly connected to each other with no physical structure blocking passage therebetween, the fluid in the first chamber 116A will not flow to the second chamber 116B. In some implementations, capillary action between the fluid and the first chamber 116A will also aid in preventing the fluid from flowing from the first chamber 116A to the second chamber 116B. In some implementations, the size of the housing 110 (e.g., the diameter of the first chamber 116A and/or the diameter of a channel 116C defined between the first chamber 116A and the second chamber 116B) can aid in preventing the fluid from flowing from the first chamber 116A to the second chamber 116B.

The base station (or other holding device) can activate the vent in order to release or relieve the air pressure generated within the housing 110. In the illustrated implementation, this is performed by removing the portion of the base station (or other holding mechanism) that is covering the plug 120 from the outside of the housing 110. For example, the base station may include a movable arm that can move between a covered position and an uncovered position. When moved to the uncovered position, air is no longer prevented from escaping through the plug 120. Thus, the air pressure within the housing 110 causes the fluid (formed from the sample and any liquid substances within the first chamber 116A) to flow from the first chamber 116A to the second chamber 116B. In the illustrated implementation, the housing 110 further defines a channel 116C that is located between the first chamber 116A and the second chamber 116B. When the vent is activated and the generated air pressure is released, the fluid will flow from the first chamber 116A, through the channel 116C, and into the second chamber 116B. Because the plug 120 is made from a hydrophobic material, the plug 120 prevents the fluid from spilling out of the device 100 through the second opening 114B.

The second chamber 116B can then be used for any desired steps of the assay. In some examples, the second chamber 116B may include additional substances needed for the specific assay being performed, such as other reagents, buffers, etc. In other implementations, the second chamber 116B can be used as a measurement chamber to obtain results. For example, if the assay being performed utilizes an optical-based analysis (such as a colorimetric analysis or a fluorescence analysis), the housing 110 (or the portion of the housing 110 that surrounds the second chamber 116B) may be formed from an optically transparent or translucent material, such that any color change can be observed. In another example, the second chamber 116B may contain a probe (such as a chemical probe, an electrical probe, etc.) that can be used to test the fluid that flows into the second chamber 116B, and generate one or more signals representative of the result of the assay. In additional or alternative implementations, the second chamber 116B can include one or more substances configured to aid in mixing the sample and the liquid reagent (or other substances).

In some implementations, the device 100 may include one or more seals positioned within the housing 110 that are designed to preserve substances used in the assay, such as reagents. For example, FIG. 1 shows the remains of a first foils seal 113A and a second foil seal 113B that have been pierced by the elongated member 150. The first foil seal 113A is located at the end of the first chamber 116A nearest the first opening 114A, and the second foil seal 113B is located at the end of the first chamber 116A nearest the channel 116C. Any reagents or other substances in the first chamber 116A can be located between the two seals, so that these substances are not exposed to the environment prior to the device 100 being used. When the elongated member 150 is inserted after being used to collect a sample, the tip 162 of the sample collection head punctures the first and second foil seals 113A and 113B so that the sample can mix with the substances, and so that the first chamber 116A is fluidly connected to the second chamber 116B. While FIG. 1 shows only the first and second foil seals 113A and 113B in their specific locations, the device 100 may include any number of seals in any number of positions.

The base station can perform other functions as well, including time and temperature control. For example, some assays require a sample to remain at a certain temperature for a certain amount of time. While the device 100 is held by the base station, the base station can be used to heat any substance within a given chamber of the device 100 to a desired temperature, and then after a desired amount of time has passed, activate the vent to cause the fluid to flow from one chamber to another chamber. The base station and the device 100 thus form a system that can be used to perform an assay.

In some implementations, the elongated member 150 can be initially inserted into the housing 110 without generating the air pressure in the first chamber 116A and the second chamber 116 (e.g., without forming the seal between the first end 112A of the housing 110 and the sealing members of the elongated member 150). The reaction within the first chamber 116A can then be performed, after which the elongated member 150 is inserted the rest of the way, to generate the air pressure. Then, the vent can be activated to cause the fluid to flow from the first chamber 116A to the second chamber 116B. In other implementations, the elongated member 150 is fully inserted and the air pressure is generated, and then the reaction in the first chamber 116A is performed.

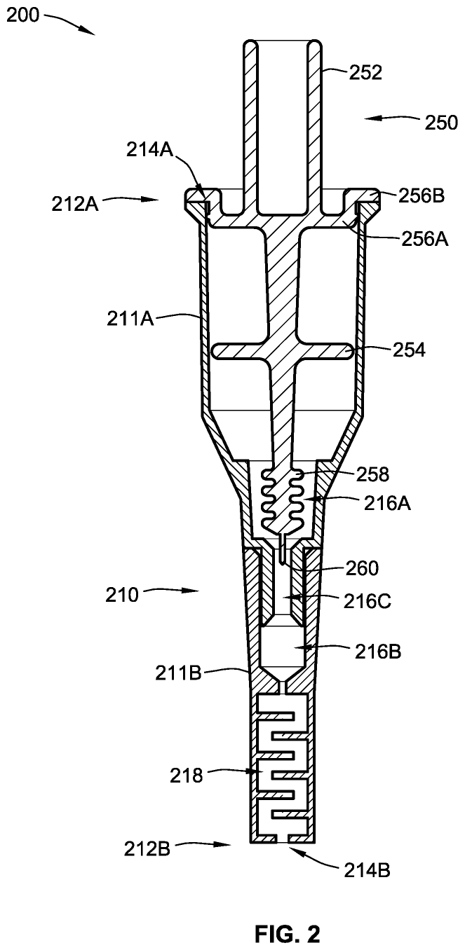

FIG. 2 is a cross-sectional view of a testing device 200, according to some implementations of the present disclosure. Testing device 200 is similar to testing device 100, but utilizes a different vent to control the flow of fluid between the chambers. Similar to device 100, device 200 includes a housing 210 formed from separate housing portions 211A and 211B coupled together. However, housing 210 could be formed from a single unitary piece in other implementations. The housing 210 includes a first opening 214A defined at a first end 212A of the housing 210, and a second opening 214B defined at a second end 212B of the housing 210.

The device 200 includes an elongated member 250 that is similar to the elongated member 150 of the device 100. The elongated member 250 includes a handle 252 and a sample collection head formed from a plurality of radially-extending ribs 258 and an axially-extending tip 260. The elongated member 250 includes a circumferential flange similar to elongated member 150. However, the circumferential flange of the elongated member 250 includes an inner flange 256A and an outer flange 256B. When the elongated member 250 is inserted into the device 200, the inner flange 256A contacts the interior of the housing 210, while the outer flange 256B contacts the housing 210 near the first end 212A of the housing 210. The contact between the inner and outer flanges 256A and 256B and the housing 210 forms a seal that prevents liquid and air from entering or exiting the housing 210 through the first opening 214A, similar to the elongated member 150. The elongated member 250 can further include an additional flange 254 that is positioned further into the first chamber 216A, that can contact the interior of the housing 210 and aid in forming the seal. Thus, the elongated member 250 includes sealing members that seal the first opening 214A when the elongated member 250 is inserted into the housing 210.

The vent of the device 200 is formed from the second opening 214B, and a channel 218 that is defined by the housing 210 between the second chamber 216B and the second opening 214B. During use, the device 200 can be placed into a base station (or other holding mechanism), prior to the elongated member 250 being inserted. The base station will have some component that seals off the second opening 214B, such as a movable arm. When the elongated member 250 is inserted into the housing 210, an air pressure is generated between the sealing members of the elongated member 250 (e.g., the inner flange 256A, the outer flange 256B, and/or the additional flange 254) and the second opening 214B. The base station can then activate the vent (for example by moving the movable arm of the base station) to release this generated air pressure. The release of the generated air pressure causes the fluid in the first chamber 216A (which generally includes the sample and one or more substances, such as reagents) to move from the first chamber 216A into the second chamber 216B. In the illustrated implementation, the housing 210 further defines a channel 216C that is located between the first chamber 216A and the second chamber 216B. When the vent is activated and the generated air pressure is released, the fluid will flow from the first chamber 216A, through the channel 216C, and into the second chamber 216B.

Because device 200 does not include the plug, the vent further includes the channel 218 defined by the housing 210. The channel 218 is defined between the second chamber 216B and the second opening 214B, and acts as an overflow channel for any excess fluid that flows out of the second chamber 216B when the generated air pressure is released. In the illustrated implementation, the channel 218 repeatedly loops back and forth on itself in a looping configuration pattern. Because of this looping configuration, the length of the channel 218 (e.g., the distance that fluid flowing through the channel 218 travels) is longer than the straight-line distance between the second chamber 216B and the second opening 214B. Thus the channel 218 can collect excess fluid from the second chamber 216B, and prevent fluid from spilling out of the device 200 through the second opening 214B.

Similar to device 100, the first chamber 216A and/or the second chamber 216B can include any number of substances needed to perform a desired array, such as reagents, buffers, etc. Moreover, device 200 can include foil seals located at different positions within the housing (such as at the ends of the first chamber 216A) in order to preserve the substances within prior to use of the device 200. Further, the housing 210 can be formed from an optically transparent or translucent material, so that color changes within the second chamber 216B can be detected (for example if using colorimetric or fluorescent analyses). The device 200 may also include one or more probes (e.g., an optical probe, a chemical probe, etc.) disposed within the second chamber 216B (or any other location of the device 200) to perform a measurement on the fluid within the second chamber 216B.

Similar to device 100, the base station can perform other functions as well, including time and temperature control. For example, some assays require a sample to remain at a certain temperature for a certain amount of time. While the device 200 is held by the base station, the base station can be used to heat any substance within a given chamber of the device 200 to a desired temperature, and then after a desired amount of time has passed, activate the vent to cause the fluid to flow from one chamber to another chamber. The base station and the device 200 thus form a system that can be used to perform an assay.

FIGS. 1 and 2 show two specific implementations of a testing device that utilizes air pressure to cause fluid to flow from a first chamber to a second chamber. However, other implementations may use different types of vent. For example, in some implementations, the vent of the device may include a movable member (such as an arm or flap that is hingedly coupled to the housing) that can be moved between a sealed position and an unsealed position. In the sealed position, the movable member covers the second opening, such that the air pressure is generated in response to the elongated member being inserted into the housing. The movable member can then be moved to the unsealed position where the second opening is uncovered. The generated air pressure will be released, and the fluid will flow from the first chamber to the second chamber.

Other implementations are contemplated that use additional or alternative mechanisms for advancing fluid from one chamber to the next. For example, the weight of a substance within the device 100 or 200 can aid in causing fluid to flow from one chamber to another. In another example, the material forming the housings 110 and/or 210 can be made from a hydrophilic material. The interaction between the fluid and the hydrophilic material can be utilized to cause the fluid to advance from one chamber to another.

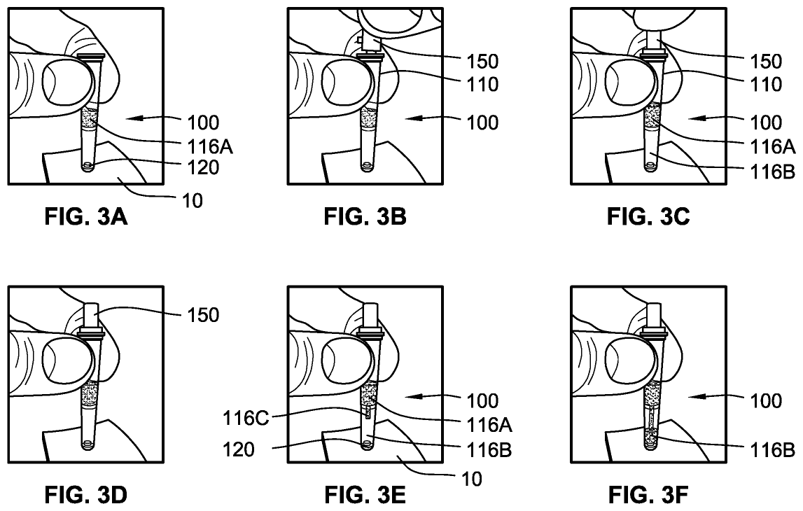

FIGS. 3A-3F show an example sequence of using the vent of device 100 to control fluid flow from a first chamber to a second chamber. In FIG. 3A, the device 100 is placed on a base 10, which covers the plug 120 and seals the interior of the housing 110. Fluid is located within the first chamber 116A. In FIG. 3B, the elongated member 150 is initially inserted into the housing 110 of the device 100. In FIG. 3C, the elongated member 150 is fully inserted into the housing 110 of the device 100, such that air pressure is generated within the first chamber 116A and the second chamber 116B. In FIG. 3D, the user has removed their finger from the top of the elongated member 150, showing that no air or liquid can escape upward through the elongated member 150. In FIG. 3E, the user has lifted the device 100 off of the base 10, and the plug 120 at the bottom of the device 100 is uncovered. Because the plug 120 is formed from a porous material, the air pressure is released, the air can flow through the plug 120. As can be seen in FIG. 3E, the fluid has begun to flow from the first chamber 116A into the channel 116C separating the first chamber 116A and the second chamber 116B. Finally, in FIG. 3F, the fluid has begun to flow into the second chamber 116B of the device 100.

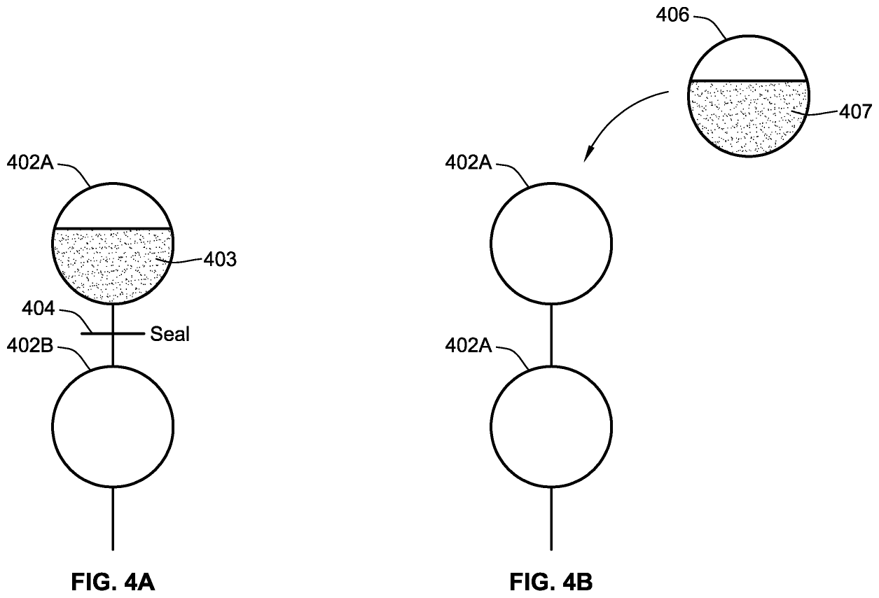

FIGS. 4A and 4B show two different implementations of adding liquid reagents in an example testing device, which could be the same as or similar to device 100 or device 200. In FIG. 4A, an amount of liquid reagent 403 has been added to a first chamber 402A (which may be similar to first chamber 116A or first chamber 216A). A seal 404 (such as a foil seal) separates the first chamber 402A from a second chamber 402B (which may be similar to second chamber 116B or second chamber 216B). Thus, the liquid reagent 403 can be added to the first chamber 402A when the device is manufactured, as the seal 404 can aid in preserving the liquid reagent 403. As discussed herein, in some implementations an additional seal can be placed on the other side of the first chamber 402A to further aid in preserving the liquid reagent 403. In FIG. 4B, an amount of liquid reagent 407 is kept in a separate reservoir 406 until the device is ready for use. At that point in time, the liquid reagent 407 can be added to the first chamber 402A. Because the liquid reagent 407 is kept in the separate reservoir 406, the seal 404 does not need to be added to the device between the first chamber 402A and the second chamber 402B when the device is manufactured.

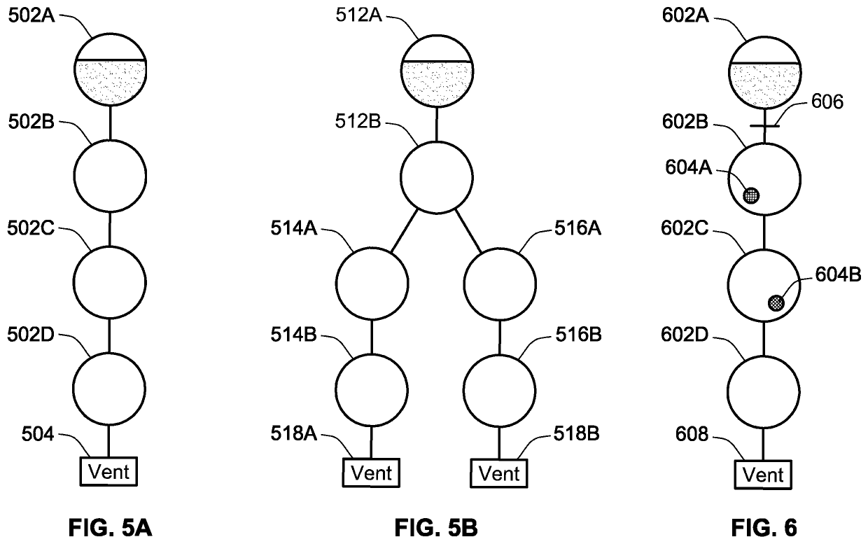

While FIGS. 1, 2, 4A, and 4B show only a linear series of two chambers in the testing device, other arrangements can also be used. FIG. 5A shows a representation of an example testing device that includes four chambers arranged in a linear fashion. The device includes a first chamber 502A, a second chamber 502B that is fluidly connected to the first chamber 502A, a third chamber 502C that is fluidly connected to the second chamber 502B, and a fourth chamber 502D that is fluidly connected to the third chamber 502C. The first chamber 502A includes a liquid reagent, and can receive the sample collection head of an elongated member. The device also includes a single vent 504. When the elongated member is inserted into the device of FIG. 5A, air pressure is generated in the chambers 502A-502D. When the vent 504 is activated, the air pressure is released, so that the fluid in the first chamber 502A (which includes the liquid reagent and the sample) can flow to the second chamber 502B, the third chamber 502C, and the fourth chamber 502D.

However, in other implementations, the vent 504 could quickly be deactivated after the fluid flows into the second chamber 502B, to prevent the fluid from flowing into the third chamber 502C and the fourth chamber 502D. After a desired amount of time, the vent 504 could again be activated to cause the fluid to flow into the third chamber 502C and the fourth chamber 502D, or only the third chamber 502C (at which point in time the vent could be deactivated, and then later re-activated to cause the fluid to flow into the fourth chamber 502D). In even further implementations, the device may include an additional vent that separately control the flow of the fluid from the second chamber 502B to the third chamber 502C, and/or from the third chamber 502C to the fourth chamber 502D. In some of these implementations, the elongated member can be removed and reinserted into the housing of the device to generate additional air pressure.

In some implementations, the amount of liquid reagent in the first chamber 502A is enough so that at least some of the fluid (the liquid reagent and the sample) is able to flow into each of the other chambers 502B-502D. In other implementations, the other chambers 502B-502D may include additional amounts of the liquid reagent (or another substance) as needed. In further implementations, the other chambers 502B-502D may be smaller than the first chamber 502A, such that an amount of liquid sufficient to fill (or partially fill) the first chamber 502A is able to fill (or partially fill) the other chambers 502B-502D.

FIG. 5B shows a representation of an example testing device with a series of chambers arranged in a parallel. The device in FIG. 5B includes a first chamber 512A, a second chamber 512B, and then two separate branching pathways. The first pathway includes a third chamber 514A and a fourth chamber 514B. The second pathway includes a third chamber 516A and a fourth chamber 516B. The device includes two separate vents 518A and 518B. Vent 518A controls flow of the fluid through the first pathway, while vent 518B controls flow of the fluid through the second pathway.

The first chamber 512A includes a liquid reagent, and can receive the sample collection head of an elongated member. Similar to the other devices described herein, when an elongated member is inserted into the device of FIG. 5B, air pressure is generated in all of the chambers. If only the vent 518A is activated, the fluid from the first chamber 512A (which includes the liquid reagent and the sample) flows into the second chamber 512B, the third chamber 514A, and the fourth chamber 514B. If only the vent 518B is activated, the fluid from the first chamber 512A flows into the second chamber 512B, the third chamber 516A, and the fourth chamber 516B.

However, if both the vents 518A and 518B are activated at the same time, the fluid from the first chamber 512A will flow through both pathways. Thus, after flowing into the second chamber 512B, some of the fluid will flow through the third chamber 514A and the fourth chamber 514B, while the rest of the fluid will flow through the third chamber 516A and the fourth chamber 515B. By placing different substances in the chambers of the two different pathways, different assays can be performed at the same time.

Base stations can be used to control the timing and temperature of the devices in FIGS. 5A and 5B as well. For example, a base station could heat the chambers of the two different pathways in the device of FIG. 5B to different temperatures, in order to perform two different assays that require different temperatures. A base station and any device disclosed herein can form a system that can be used to perform an assay.

FIG. 6 shows a representation of an example testing device with various different substances in separate chambers. The testing device includes a first chamber 602A with a liquid reagent contained therein, a second chamber 602B with a first lypholization bead 604A contained therein, a third chamber 602C with a second lypholization bead 604B contained therein, and a fourth chamber 602D. A seal 606 can be placed in between the first chamber 602A and the second chamber 602B. A vent 608 is used to control the flow of a fluid (e.g., a sample and the liquid reagent) from the first chamber 602A into the other chambers. The chambers 602A-602D can be used to perform a multi-step reaction (e.g., a process that includes multiple mixing and incubating steps). The chambers 602A-602D could also be used to perform different reactions.

Various other arrangements of chambers are also contemplated. In one example, two different pathways could re-join each other, with fluid from both pathways flowing into a single subsequent chamber. In another example, different pathways within a single testing device may include separate initial chambers. An elongated member with multiple sample collections heads can be inserted into the device so that each sample collection head is disposed within its in respective initial chamber, each of which containing a desired reagent. In a further example, the testing devices can be designed so that fluid initially only flows into one chamber and/or pathway, but once that chamber and/or pathway is full, the fluid begins flowing into a different chamber and/or pathway.

In another example, a testing device could implement conditional pathways based on various signals from the chambers. For example, it may be desirable to perform a first subsequent assay after one result in an initial assay, but a second subsequent assay after a different result in the initial assay. In reference to the device of FIG. 5B, an initial result of an assay can be measured in the second chamber 512B. Depending on the result, different vents can be activated to cause the fluid to flow either into the first pathway (which includes the third chamber 514A and the fourth chamber 514B), or the second pathway (which includes the third chamber 516A and the fourth chamber 516B). The two separate pathways can be designed to perform different assays. The conditional branching can be based on specific detected assay results (such as a specific color change or electrical signal), or on characteristics of a fluid within a given chamber (such as viscosity, turbidity, etc.). Generally, any of the testing devices described herein can be formed using any combination of linear paths and/or parallel paths. Flow can be controlled individually between each pair of chambers, or multiple pairs of chambers can be controlled simultaneously.

In general, a base station used with any testing device disclosed herein can have any components necessary to implement the various assays. For example, the base station can include a variety of sensors to detect outcomes of the assays, and/or the flow of the fluid. These sensors could include image sensors (such as a camera), optical sensors (such as a light emitting diode and a photodiode), and other sensors. The base station could also include a microcontroller configured to process results and control the temperature, timing, and flow of the device. The base station could further include a variety of different physical mechanisms to operate the vents of the devices. The physical mechanisms could include solenoids controlled by input/output pins of the microcontroller. If a vent needs to be activate to cause fluid to flow, a solenoid could be activated or deactivated (depending on the design of the base station and/or the solenoid) to unblock a plug (such as plug 120) or an opening (such as the second opening 214B). In some implementations, the base station could also include a mechanism to re-generate air pressure in the device. For example, the device could include an air input port that fluidly connects a given chamber to the exterior of the housing. When the device is inserted into the base station, the air input port could be coupled to an air source, so that the base station can pump air back into the chamber of the device in order to re-generate the air pressure within the device. The base station and any of the devices disclosed herein can form a system used to perform an assay.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A device for performing an assay, the device comprising:
a housing having a first end and a second end, the housing defining a first opening at the first end, a first chamber, and a second chamber, the first chamber being fluidly connected to (i) the second chamber, and (ii) an exterior of the housing via the first opening;
an elongated member configured to be received through the first opening such that the elongated member is least partially disposed within the first chamber; and
a vent configured to aid in controlling flow of a fluid from the first chamber of the housing to the second chamber of the housing.

2. The device of claim 1, wherein an air pressure is generated in the first chamber and the second chamber in response to the elongated member being received through the first opening of the housing.

3. The device of claim 2, wherein in response to the vent being activated, the air pressure generated in the first chamber and the second chamber is released, to thereby cause the fluid to flow from the first chamber to the second chamber.

4. The device of claim 3, in combination with a base station, the base station being configured to activate the vent to release the generated air pressure in the first chamber and the second chamber.

5. The device of claim 4, wherein the vent includes a second opening defined by the housing at the second end thereof, the second chamber being fluidly connected to the exterior of the housing via the second opening.

6. The device of claim 5, wherein:
the base station is configured to seal the second opening before the elongated member is received through the first opening of the housing; and
the base station is configured to unseal the second opening after the elongated member is received through the first opening of the housing, to thereby cause the fluid to flow from the first chamber to the second chamber.

7. The device of claim 5, wherein the vent includes a plug positioned in the second opening of the housing, the plug being configured to allow air to pass therethrough.

8. The device of claim 7, wherein:
the base station is configured to seal the plug before the elongated member is received through the first opening of the housing; and
the base station is configured to unseal the plug after the elongated member is received through the first opening of the housing, to thereby cause the fluid to flow from the first chamber to the second chamber.

9. The device of any one of claims 2 to 8, wherein the elongated member includes one or more sealing members configured to contact an interior of the housing in response to the elongated member being received by the housing.

10. The device of claim 9, wherein the air pressure generated in the first chamber and the second chamber is generated between the one or more sealing members of the elongated member, and the vent.

11. The device of any one of claims 1 to 10, wherein the vent includes a second opening defined by the housing at the second end thereof, the second chamber being fluidly connected to the exterior of the housing via the second opening.

12. The device of claim 11, wherein the vent further includes a channel defined by the housing between the second chamber and the second opening, the channel being configured to collect excess fluid from the first chamber in response to the fluid flowing from the first chamber to the second chamber.

13. The device of claim 12, wherein the channel has a looping configuration.

14. The device of claim 12 or claim 13, wherein a length of the channel is greater than a straight-line distance between the second chamber and the second opening.

15. The device of any one of claims 12 to 14, wherein the vent includes a plug positioned in the second opening of the housing, the plug being configured to allow air to pass therethrough.

16. The device of claim 15, wherein the plug is formed from a porous hydrophobic material.

17. The device of any one of claims 12 to 16, wherein the vent includes a movable member disposed in the second opening, the movable member being configured to move between a sealed configuration and an unsealed configuration.

18. The device of claim 17, wherein the fluid is configured to flow from the first chamber to the second chamber at least partially in response to the movable member moving from the sealed configuration to the unsealed configuration.

19. The device of any one of claims 1 to 18, in combination with a base station, the base station being configured to actuate the vent to cause the fluid to flow from the first chamber to the second chamber.

20. The device of any one of claims 1 to 19, wherein the housing is formed from an optically transparent material.

21. The device of any one of claims 1 to 20, further comprising a probe disposed in the first chamber or the second chamber.

22. The device of claim 21, wherein the probe is a chemical probe or an electrical probe.

23. The device of any one of claims 1 to 22, wherein the housing further defines a third chamber that is fluidly connected to the first chamber.

24. The device of claim 23, wherein the vent is configured to aid in controlling flow of the fluid from the first chamber to the third chamber.

25. The device of claim 24, wherein:
an air pressure is generated in the first chamber, the second chamber, and the third chamber in response to the elongated member being received through the first opening of the housing; and
in response to the vent being activated, the generated air pressure is released, to thereby cause the fluid to flow from the first chamber to both the second chamber and the third chamber.

26. The device of claim 23, further comprising an additional vent configured to aid in controlling flow of the fluid from the first chamber to the third chamber.

27. The device of any one of claims 1 to 26, wherein the housing further defines a third chamber that is fluidly connected to the second chamber.

28. The device of claim 27, wherein the vent is further configured to aid in controlling flow of the fluid from the second chamber to the third chamber.

29. The device of claim 27, further comprising an additional vent configured to aid in controlling the flow of the fluid from the second chamber to the third chamber.

30. The device of any one of claims 1 to 29, wherein the first chamber, the second chamber, or both, include one or more reagents.

31. The device of claim 30, wherein the elongated member is configured to collect a sample to be tested in the assay, and wherein the sample is configured to react with the one or more reagents in the first chamber in response to the elongated member being received through the first opening.

32. The device of any one of claims 1 to 31, wherein a volume of the fluid in the first chamber is such that gravity does not cause the fluid to flow from the first chamber to the second chamber.

33. The device of any one of claims 1 to 32, wherein the housing is sized to aid in preventing the fluid from flowing from the first chamber to the second chamber.

34. The device of claim 33, wherein the housing further defines a channel between the first chamber and the second chamber, the channel having a diameter configured to aid in preventing the fluid from flowing from the first chamber to the second chamber.

35. A device for performing an assay, the device comprising:
a housing having a first end and a second end, the housing defining a first opening at the first end, a first chamber, and a second chamber, the first chamber being fluidly connected to (i) the second chamber, and (ii) an exterior of the housing via the first opening;
an elongated member configured to be received through the first opening such that the elongated member is least partially disposed within the first chamber, the elongated member aiding in generating an air pressure in the first chamber and the second chamber; and
a vent configured to aid in controlling flow of a fluid from the first chamber of the housing to the second chamber of the housing, the vent being configured to be activated to release the air pressure generated in the first chamber and the second chamber, to thereby cause the fluid to flow from the first chamber to the second chamber.

36. A system for performing an assay, the system comprising:
a device including:
a housing having a first end and a second end, the housing defining a first opening at the first end, a first chamber, and a second chamber, the first chamber being fluidly connected to (i) the second chamber, and (ii) an exterior of the housing via the first opening;
an elongated member configured to be received through the first opening such that the elongated member is least partially disposed within the first chamber; and
a vent configured to aid in controlling flow of a fluid from the first chamber of the housing to the second chamber of the housing; and
a base station configured to receive the housing of the device therein, the base station being further configured to activate the vent to cause the fluid to flow from the first chamber to the second chamber.

37. The system of claim 36, wherein an air pressure is generated in the first chamber and the second chamber in response to the elongated member being received through the first opening of the housing.

38. The system of claim 37, wherein in response to the vent being activated by the base station, the air pressure generated in the first chamber and the second chamber is released, to thereby cause the fluid to flow from the first chamber to the second chamber.

39. A system for performing an assay, the system comprising:
a device including:
a housing having a first end and a second end, the housing defining a first opening at the first end, a first chamber, and a second chamber, the first chamber being fluidly connected to (i) the second chamber, and (ii) an exterior of the housing via the first opening;
an elongated member configured to be received through the first opening such that the elongated member is least partially disposed within the first chamber, the elongated member aiding in generating an air pressure in the first chamber and the second chamber; and
a vent configured to aid in controlling flow of a fluid from the first chamber of the housing to the second chamber of the housing, the vent being configured to be activated to release the air pressure generated in the first chamber and the second chamber, to thereby cause the fluid to flow from the first chamber to the second chamber; and
a base station configured to receive the housing of the device therein, the base station being further configured to activate the vent to cause the fluid to flow from the first chamber to the second chamber.
